(19) United States
(12) Patent Application Publication
(10) Pub. No.: US 2003/0012825 A1
(43) Pub. Date: Jan. 16, 2003

(54) Metallized molecule therapies

(76) Inventor: Charles Kapper, Milwaukee, WI (US)

Correspondence Address: Charles Kapper, 5464 N. Port Washington Road, Apt. A125, Milwaukee, WI, 53217, US

(21) Appl. No.: 09/900,973

(22) Filed: Jul. 9, 2001

Related U.S. Application Data
(60) Provisional application No. 60/217,126, filed on Jul. 10, 2000.
(60) Provisional application No. 60/283,641, filed on Apr. 16, 2001.

(51) Int. Cl. A61K 9/00; A61K 33/34; A01N 59/20

(52) U.S. Cl. 424/630; 424/400

(57) ABSTRACT

Five pharmaceutical inventions are synthesized by administering metallic substances, preferably immunoactive copper in the form of tyrosinase, with the optional use of a composition consisting of CoEnzyme Q1 to Q10, a multivitamin, and amino acids. The composition artificially maintains the presence of metallic substances beyond normal metabolism time in the tissues, organs, arteries, veins or body fluids of humans, animals, agricultural plant fibers, leafs, or root systems. The initialization of therapy utilizes radiological, X-Ray, MRI, PET or ultrasonic imaging to establish maximum dilation, followed by a micromolar metallic assay, such that continued dosages are administered to maintain the static proportional micromolar amount of the metal in the blood and urine. Five embodiments: (1) Immunoamplification by dilation of the thymus. (2) Immunoamplification by ingestion of enriched agricultural plants. (3) Prebirth Immunoamplification for poultry eggs. (4) A vasodilative treatment for seizures, spasms, excessive EEG pulses, epilepsy, vasoconstrictive headaches, tourette's syndrome, or any disorder resulting from low brain copper levels. (5) A method to reclassify plants, based on the distribution of metals to determine suitability as immunoamplification agents. Typical Immunoamplification uses: presurgical treatment for donor/acceptor organ transplant, AIDS, HIV, or, to increase the T-Lymphocyte helper cells.

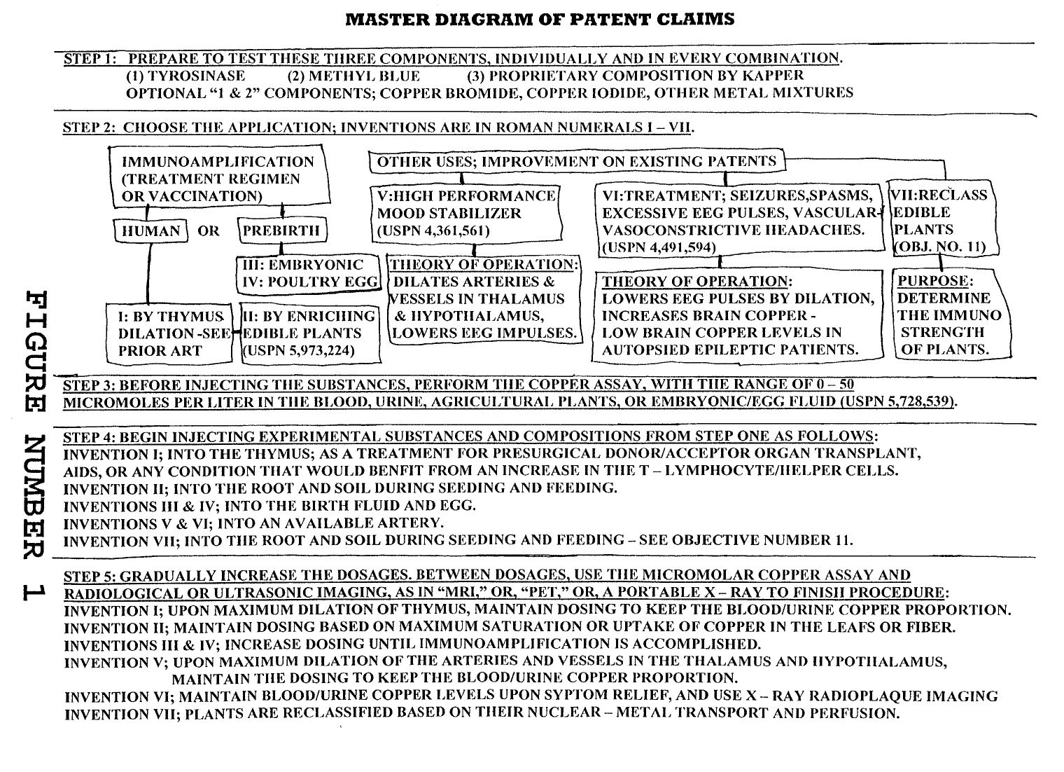

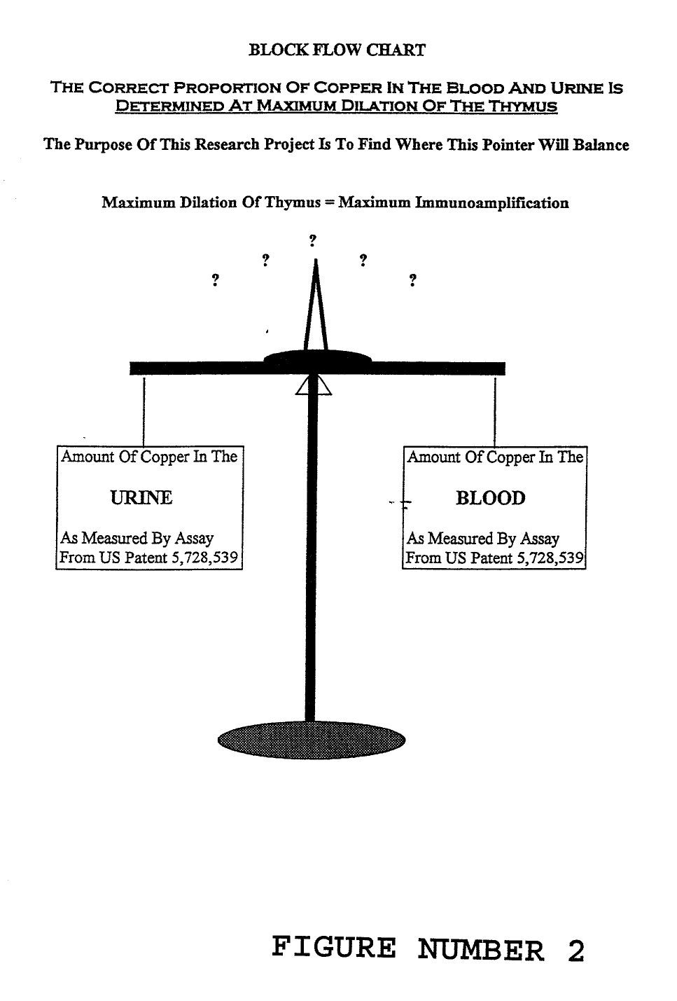

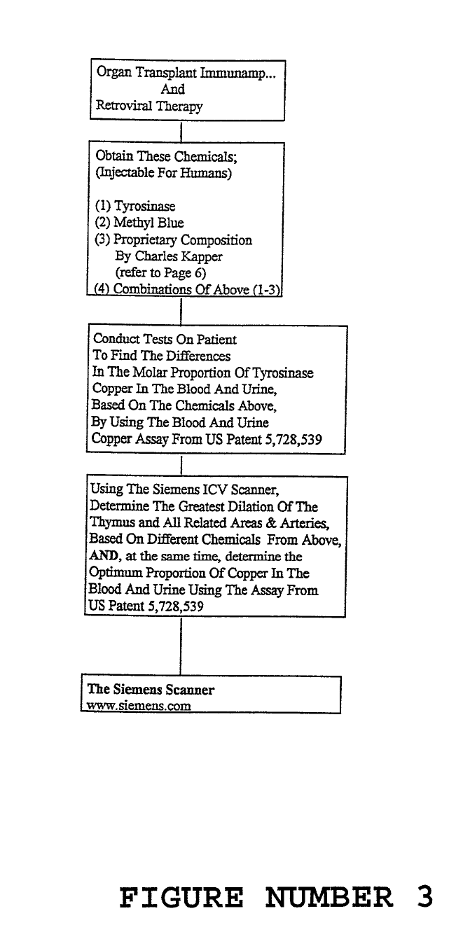

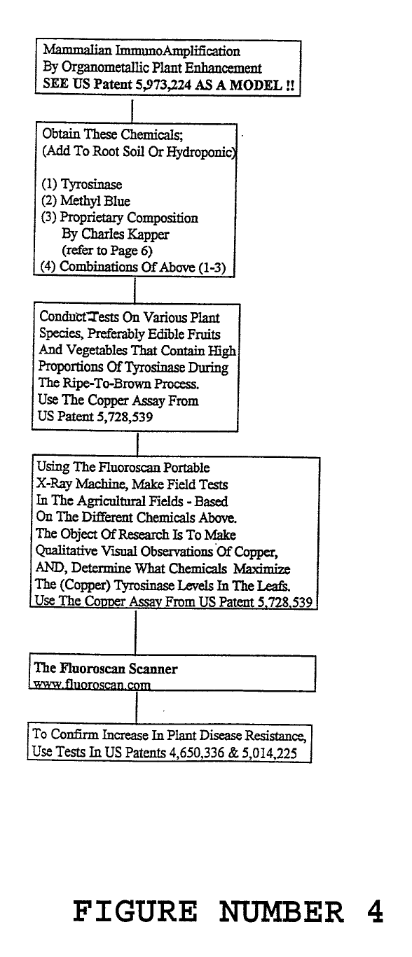

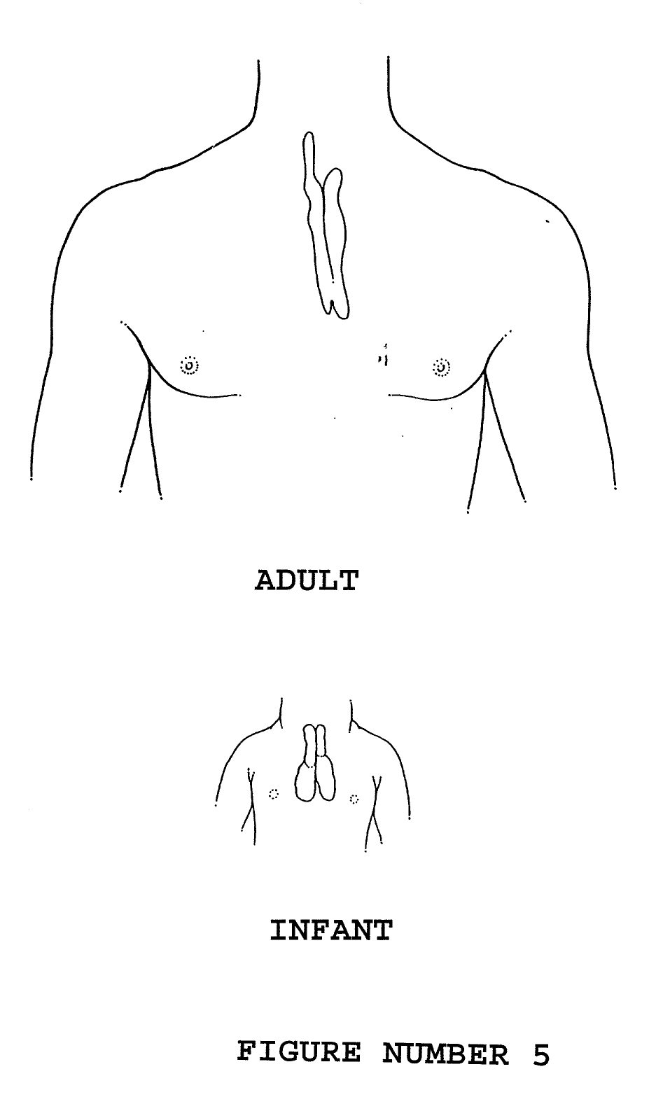

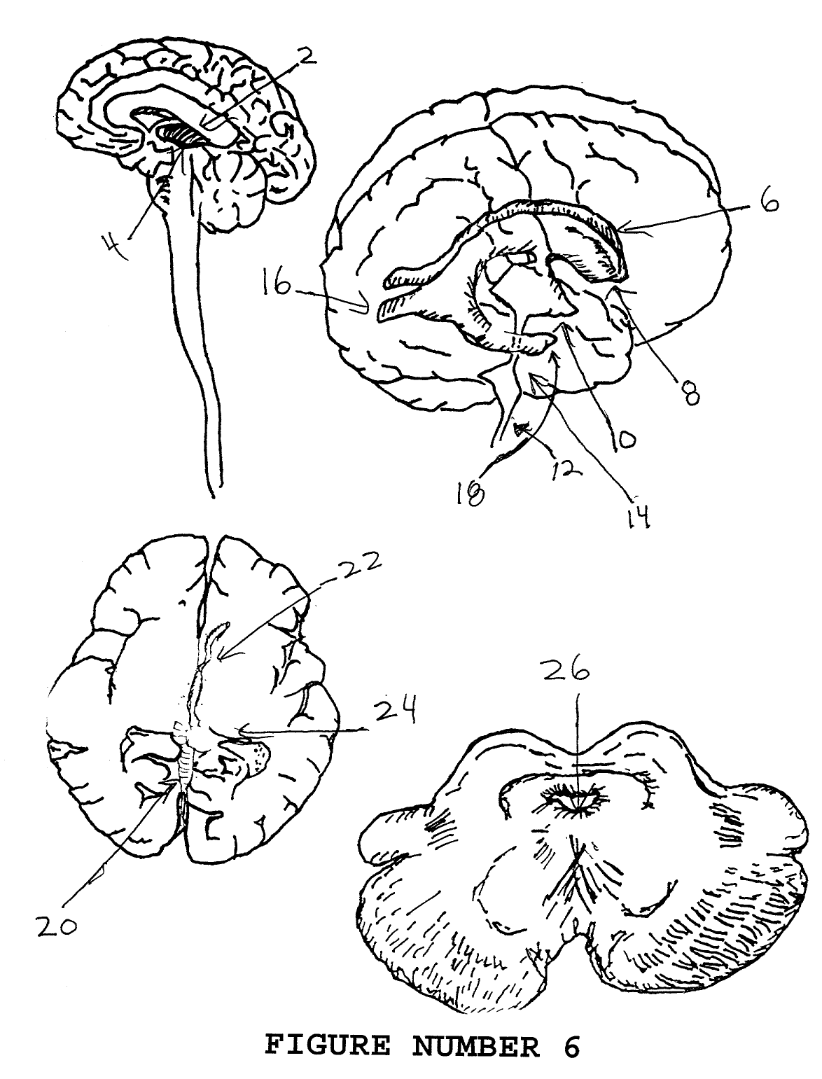

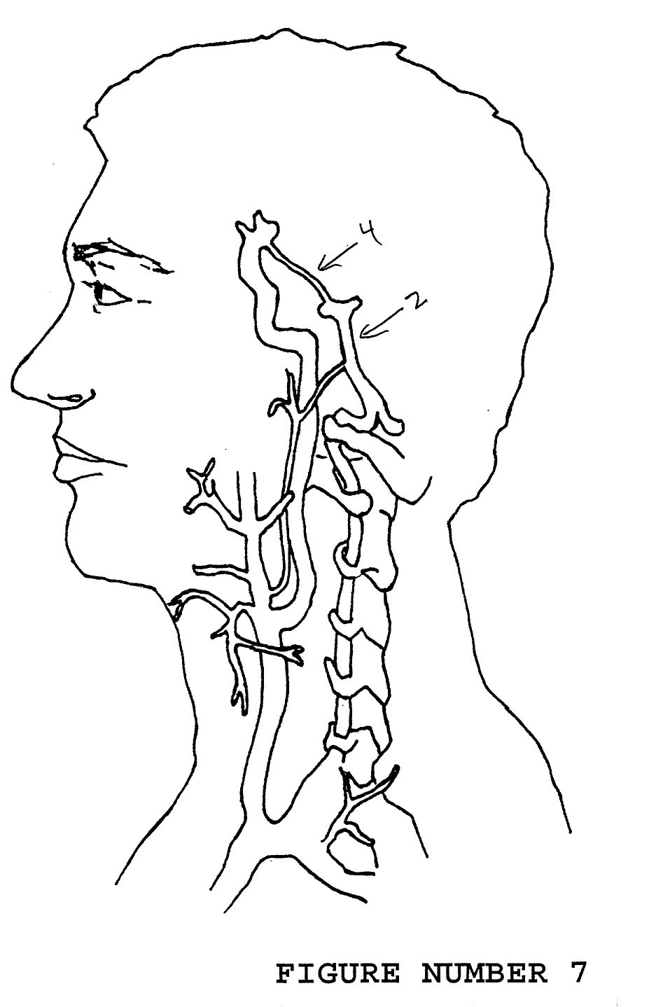

### PRIOR ART

In addition to the scientific literature and patent documents, there are four separate U.S. Patent Office filings related to this invention that have been submitted by inventor Kapper, and necessitate explanation as prior art. These are:

A Provisional Application, filed Jul. 10, 2000. Serial No. 60/217,126

A Treatise, deposited Jul. 22, 2000 with the “Document Disclosure” program. Ser. No. 477,325

A Provisional Application, filed Apr. 16, 2001. Serial No. 60/283,641

This Regular U.S. Application; which incorporates the abovementioned three previous filings.

In order to avoid confusion regarding which particular patent office filing the text in this specification refers to, the phrase “this application” will refer to this regular U.S. application. Obviously, this application has the most recent date.

A Provisional Application; was filed on Jul. 10, 2000, Serial No. 60/217,126 by inventor Charles R. Kapper, with the title “Immunoamplification Utilizing Radiological Administration Of Metalloproteins.”

Page 3 of this Provisional Application; refers to the concept of establishing a maintenance-dosage level of copper to dilate or enlarge the thymus. The procedure is as follows: Copper substances are injected into the thymus with gradually increased dosages, based on the molar-copper amount.

As the gradual increase of serum copper by injection takes place, two measurements are made after each increase of copper dosage. First, the size of the thymus is monitored by utilizing either Magnetic-Resonance-Imaging, Positron-Emissive Tomography, or ultrasonic-imaging technology. Secondly, and after each injection, a micromolar copper assay is performed on the blood and urine. The proper dosage of copper is based on when the thymus has achieved maximum dilation or enlargement. The amount and frequency of molar copper to be injected is based on the concept that the thymus will remain dilated, as long as the exact molar copper proportion in the blood and urine remains the same, and corresponds to when the thymus has achieved maximum dilation or enlargement. This concept of a “balanced-blood—and—urine-molar-copper-proportion” to achieve maximum dilation or enlargement of the thymus is illustrated on page 3 of this provisional application.

Pages 1, 4, and 6 of this Provisional Application; consists of the title page, a page that contains two block-diagrams that restate and compliment the previous subject matter from the previously-discussed page 3, and a page that discusses some general pharmacological properties (page 6). These pages refer to the embodiment(s) of this invention that claim: a presurgical treatment for organ transplant immunoamplification for both the donor and acceptor, a treatment for retroviral illnesses, and, another embodiment, which is an improvement over U.S. Pat. No. 5,973,224. The improvement over U.S. Pat. No. 5,973,224 consists of immunoamplification by the enrichment of agricultural foodstuffs by utilizing the copper substances and compositions by inventor Kapper as plant fertilizer. In the plant fertilizer embodiment, the aim of the procedure is to find the perfect substance(s) or composition that will cause the greatest molar amount of copper to be left in the plant, after it has been treated with the proprietary compositions. The substances are to be added to the soil when the plant seed is initially placed in the ground to commence growing, or, on plants that have already been growing for a while, and are currently producing edible foodstuffs. These copper substances and compositions for both of these embodiments are contained on page 4, and are a set of proprietary compositions and specific individual substances by inventor Kapper. They are also found in detail on page 6. They are evaluated individually and in every combination. The four basic groups are: (1) Tyrosinase, (2) Methyl Blue, (3) a proprietary composition by inventor Kapper that consists of Tyrosine, Coenzyme Q10, tryptophane, Aromatic Amino Acids, Phenylalanine, other Amino Acids, and the use of a vitamin Base. (4) Combinations of the three previously-mentioned substances or compositions.

Page 6 also refers to two separate features and claims. First, the concept of using the pharmaceutical compounds by Kapper to create a sustained and increased presence of copper in the thymus for the purpose of enlarging or dilating the thymus for immunoamplification. The second concept refers to using the same set of copper substances and compositions to increase the copper levels in the brain. At the same time, the composition takes advantage of the fact that tyrosine has the ability to cross the blood-brain barrier. This supports yet another claimed feature, which is the unique arterial transport path (Merck Index 1983).

When the embodiment known as immunoamplification by agricultural fertilizer enrichment is used, two separate measurements are gathered to assay the amount of copper in the plants, and are performed after each increase in copper dosing. First, the non-radiological, micromolar chemical-assay is used to find the level of molar copper in the different areas of the plant. Secondly, the radiology system is then used to measure the distribution of copper in the agricultural plants. This makes use of a portable X-ray machine that can be used in a farm field. In this application, it would not be possible to use conventional and large Magnetic-Resonance-Imaging, or, Positron-Emissive-Tomography equipment. Therefore, because it is not necessary to use such expensive and large radiological equipment, a substantial cost-savings is realized.

Page 5 of this Provisional Application; Section 1 of this page refers to the concept of using radiology technology to monitor the dilation or enlargement of the thymus from the sustained level of molar copper for immunoamplification purposes, and, the concept of using the proprietary and experimental compositions by inventor Kapper to sustain and increase molar-copper levels in the brain. This page also discloses the use of the micromolar copper assay to ascertain the proportion of molar copper in the blood and urine. The purpose of monitoring these molar-copper levels, is that as long as this proportion is maintained by the copper injections, it is not necessary to perform multiple radiological exposures to confirm that the thymus is dilated or enlarged.

Page 7 of this Provisional Application; refers to the concept of increasing molar-copper levels in the brain for purpose of lowering EEG brain-electrical activity, and the concept of adjusting these copper levels for the purpose of optimizing the use of radiological equipment.

A “Document Disclosure” was Filed On Jul. 22, 2000, Ser. No. 477,325, by inventor Charles R. Kapper.

This document contains all of the documents found in the previous provisional application, filed on Jul. 10, 2000. However, in addition to the material found in the provisional application, this Document Disclosure also claims that any substance or composition that contains: (1) A copper atom, and/or, (2) An atom from the Group Ib periodic table and/or (3) Atom(s) of gold or silver are included in this invention.

A Provisional Application; was filed on Apr. 16, 2001 Serial No. 60/283,641 by inventor Charles R. Kapper, with the title “Vaccination Or Immunoamplification By Dilation Of Thymus With Polynuclear Metabolites And Radiological Monitoring.”

This provisional application contains two embodiments found in the provisional application from Jul. 10, 2000. It also contains the claims from the “Document Disclosure” filed on Jul. 22, 2000, which claims the invention may utilize any substance or composition that contains: (1) A copper atom, and/or, (2) An atom from the Group Ib periodic table and/or (3) Atoms of gold or silver.

In addition to the abovementioned elements, the new material claims that:

The immunoamplification of the invention can be used in a vaccination capacity, in addition to treating an existing illness.

Vitamin A Acetate—retinoids can also be used to dilate the thymus.

The medicinal applications of this therapy can also include aids, cancer, leukemia, bone marrow transplant, and tumors. Other applications include any abnormal medical condition, sickness, or malady that can be treated by an amplified immune system, such that the amplified immune system is induced and/or synthesized by a dilated or enlarged thymus by the use of this invention treatment. These include, but are not limited to viral, retroviral, autoimmune, bacterial, infectious diseases, fungi, insect and/or vermin carrying, tropical or climatic-based, rheumatic family of illnesses, and, utilize any type of transport and/or exposure method to humans that include, but are not limited to parasitic, airborne, body fluid contamination, or genetic disposition.

In the thymus, for the purpose of dilation, to cause immunoamplification.

Agricultural plants, to enrich foodstuffs for immunoamplification.

The principle of dilating the thymus for immunoamplification is used in this disclosure, and is based on U.S. Pat. Nos. 5,234,683, -5,114,708, and 4,215,137.

However, U.S. Pat. Nos. 5,234,683, -5,114,708 and 4,215,137 do not use the copper-containing substance tyrosinase, or any other form of copper for thymus dilation. Tyrosinase and copper compounds are not known as vasodilators.

The belief by inventor Kapper that tyrosinase and copper-containing substances are vasodilators is partially based on the two articles in the IDS by Cappelli-Bigazzi and Stein. Further scientific support is suggested by several patents mentioned in the prior art section. These patents were issued to a Dr. Sorenson in Little Rock, Ark.; U.S. Pat. No. 4,670,428, U.S. Pat. No. 4,757,059, and U.S. Pat. No. 4,758,554. The Sorenson patents discuss the use of copper compounds for convulsions and epilepsy.

Inventor Kapper has two theories about Tyrosinase being a vasodilator. He believes that convulsions, seizures, and involuntary muscle movements are at least and in part due to excessive electrical impulses in the brain. He also believes that excessive vasoconstriction is a cause of excessive electrical impulses. Therefore, if copper is a successful treatment for these symptoms, it probably is a vasodilator. It's also obvious that the presence of copper in the bloodstream changes the “steep gradients” of electrical charges in the muscles and brain, in the sense that the relative strengths of these charges are “smoothed out.” Therefore, the copper is providing a decreased path of resistance to the electrical charges. Kapper also points out that the use of amino acids as vasodilators, such as tyrosine, is very well established in patent literature. The perfusion, transport, and metabolic mechanism of tyrosine has the ability to pass the blood-brain barrier (Merck 1983). This suggests the strong possibility that the cellular walls spread out to increase the diameter of the arteries. Furthermore, U.S. Pat. No. 4,491,594 discusses the use of a substance known as Coenzyme Q10, a substance that changes cellular respiration.

Inventor Kapper theorizes a mechanism of operation, wherein changes in cellular respiration caused by Coenzyme Q10 induces vasodilation by widening the artery diameter—by changes in the size of the cellular wall of the artery during cellular respiration.

Kapper also clearly points out that the use of tyrosinase or copper-containing substances is well established in both patent documents and scientific literature for organ transplantation, tumor treatment, cancer, leukemia and certain retroviral illnesses. The references in this patent application are only a very small sampling of available citations.

U.S. Pat. No. 3,170,836 by Victor discloses general considerations of preparing liquid and injectable copper-containing substances.

U.S. Pat. No. 3,982,999 by Kharasch discloses the use of tyrosinase to inhibit L1210 mouse leukemia. Tyrosinase is used by inventor Kapper in this disclosure to treat leukemia, but uses a different mechanism—of—operation than Kharasch. Kharasch also discloses the preparation of intravenous solutions from dried preparations, typically being 500 milligrams per 10 milliliters of solution, with the proper dosing being 15 to 100 milligrams per kilogram of body weight. These injections last from five days to two weeks. Kharasch also discloses the storage and refrigeration requirements. The shelf life could be several months, based on 1 milligram of dried preparation per milliliter of solution. The copper chelate is added to the tyrosinase.

U.S. Pat. No. 4,215,137 by Dobson et al discloses medicating the thymus of a mouse to induce thymus enlargement and the related benefits to the immune system. Inventor Kapper hereby incorporates the following clinical indications by Dobson into the performance and claim specifications of this invention disclosure: Natural cellular immunity in the normal host is known to be initiated by contact between an invading foreign antigen (bacteria, virus, protozoa, neoplastic cell, etc.) and thymus-derived lymphocytes (T-cells) which, when stimulated by foreign antigen, release soluble factors (lymphokines) into circulation. These factors or enzymes produced by the T-cells in turn activate the macrophages which destroy the invading organism by a process of phagocytosis, followed by a direct attack on the organism by enzymes that dissolve the invading organism (lysosomal enzymes). Organisms such as tubercle bacilli and leprosy bacilli survive phagocytosis, and even multiply within nonactivated macrophages. The activated macrophage however, destroys these organisms by increased concentrations of lysosomes and lysosomal enzymes. Bennett et al., J. Transplantation, 5, 996-1000.

U.S. Pat. No. 4,361,561 by Naylor discloses the use of a copper-containing substance for treatment of bipolar disorder, and discusses a suitable dosage regimen, which is important for toxicity and safety considerations. Naylor uses Methylthionine chloride, also known as Methylene Blue, and administers a dosage rate of from 0.1 to 10 milligram per kilogram of bodyweight per patient per day.

U.S. Pat. No. 4,491,594 by Ogawa et al discloses the use of Coenzyme Q10 for treating convulsions and epilepsy, with important toxicity and safety information about Coenzyme Q10 dosing. Ogawa refers to the treatment of some of the same disorders contained in this disclosure. However, unlike this disclosure, the Ogawa patent does not use arterial dilation as a mechanism of treatment in using Coenzyme Q10 as a pharmacological agent. Ogawa states that the dosage can be between 10 to 1000 milligrams per day, and further states that Coenzyme Q10 is a yellow or orange powder, which is soluble in chloroform, benzene, carbon tetrachloride, acetone and ether, but insoluble in ethanol, water and methanol, with the CoQ10 melting point being about 48.degree. C. What is also significant about the Ogawa patent in relation to the proprietary composition by inventor Kapper, is that Ogawa indicates a lowering of EEG—electroencephalogram brain activity. This supports the claim by inventor Kapper in this patent application that it is possible to pharmacologically induce vasodilation and sustain the presence of copper at the same time.

U.S. Pat. No. 4,650,336 by Moll discloses a standardized laboratory procedure to measure fluorescence in agricultural plants. This is important to this disclosure because in order to image metals in a plant using a conventional optical microscope, a substantial amount of fluorescence must be generated from the plant sample, by exposing the plant to the excitation of ultraviolet radiation and the use of dye-markers. This is different than the radiological method suggested in this disclosure, but could be utilized if radiology equipment was not available.

U.S. Pat. No. 4,670,428 by Sorenson discloses the use of copper for treating convulsions and epilepsy, with important toxicity and safety information about copper dosing. Sorenson refers to the treatment of some of the same disorders contained in this invention disclosure. However, unlike this disclosure, the Sorenson patent does not use arterial dilation as a mechanism of treatment in using copper as a pharmacological agent. Sorenson states that copper is a normal component of the human brain, which contains about 370 mg of copper per gram of tissue ash. This amount of tissue copper ranks second only to the amount found in the liver, the storage organ for copper. According to Sorenson, a variety of brain pathologic disorders accompanied by convulsive seizures are associated with abnormally-low copper levels in the brain. Serum copper is elevated in epileptic patients, but brain copper levels are markedly reduced in autopsied epileptics. Therefore, unlike Sorenson, a primary object of this invention disclosure is to increase the amount of serum copper in the brain, and, at the same time, decrease the electrical activity in the brain by “smoothing out” the electrical gradient of EEG activity within the brain, as in “shortening the distance” of the peaks and valleys of the electrical activity. Tyrosinase is a copper-containing substance, and, therefore, the invention submitted in this disclosure is further accomplished by the unique property of tyrosine being able to cross the blood-brain barrier (Merck 1983). Getting back to Sorenson, he further states that anticonvulsant activity is initiated with Cu(II) (salicylate) .sub.2 in preventing the Metrazol-induced seizure after giving 100 milligrams per kilogram of bodyweight subcutaneously and in preventing the Maximal Electroshock-induced seizure after giving 600 milligrams per kilogram of bodyweight subcutaneously. Some of these compounds were found to have anticonvulsant activity at doses less than 30 milligrams per kilogram of bodyweight, and for prolonged periods of up to 6 to 8 hours post injection.

U.S. Pat. No. 4,757,059 by Sorenson discloses the use of copper for treating convulsions and epilepsy, with important toxicity and safety information about copper dosing. This Sorenson patent refers to the treatment of some of the same disorders contained in this invention disclosure. However, unlike this disclosure, the Sorenson patent does not use arterial dilation as a mechanism of treatment when using copper as a pharmaceutical agent.

U.S. Pat. No. 4,758,554 by Sorenson discloses the use of copper for treating convulsions and epilepsy, with important toxicity and safety information about copper dosing. This Sorenson patent refers to the treatment of some of the same disorders contained in this disclosure. However, unlike this disclosure, the Sorenson patent does not use arterial dilation as a mechanism of treatment when using copper as a pharmaceutical agent.

U.S. Pat. No. 4,952,607 by Sorenson et al discloses the use of copper to treat cancer, but uses a different mechanism than this invention disclosure.

U.S. Pat. No. 5,011,858 by Langsjoen et al discloses the use of Coenzyme Q10 for treatment of AIDS or other retroviral illnesses. Coenzyme Q10 is an essential component within the proprietary composition by inventor Kapper.

U.S. Pat. No. 5,014,225 by Vidaver et al discloses a standardized laboratory procedure to measure fluorescence in agricultural plants. This is important to this disclosure because an order to image metals in a plant using a conventional optical microscope, a substantial amount of fluorescence must be generated from the plant sample, by exposing the plant to the excitation of ultraviolet radiation and the use of dye-markers. This is different than the radiological method suggested in this disclosure, but could be utilized if radiology equipment was not available

U.S. Pat. No. 5,114,708 by Hunter et al discloses the concept of amplifying the immune system by increasing the size of the thymus. This disclosure increases the size of the thymus, but uses a different set of pharmacological agents, and uses a radiological technique known as Magnetic-Resonance-Imaging or ultrasonic detection to initialize the therapy, and to guard against dangerous copper toxicity. Hunter states that the cure for a person with AIDS will probably require one agent to eliminate the virus and other agents to cause the body to replace T cells that have been killed by the virus. Hunter also mentions the considerations in his invention regarding immunoamplification to combat poultry diseases in young animals with nondeveloped or undeveloped immune systems.

U.S. Pat. No. 5,234,683 by Hunter et al discloses the concept of amplifying the immune system by increasing the size of the thymus. Inventor Kapper also uses a mechanism that increases the size of the thymus, but uses a different set of pharmacological agents, and, uses a radiological technique known as Magnetic-Resonance-Imaging or ultrasonic imaging to initialize therapy and guard against dangerous copper toxicity.

U.S. Pat. No. 5,728,539 by Nonobe et al discloses a laboratory method for measuring small amounts of copper in the blood and urine. Such a procedure is used by Kapper to practice the invention in this disclosure. Nonobe states that Copper is present in the blood only in minute amounts, with a normal value of 12.9-21.1 micromoles per liter for males, and 16.2-25.0. micromoles per liter for females. Nonobe further states in routine medical care, a greater problem occurs when a low value, rather than a high value of serum copper is shown, and is manifested by Wilson's disease and Kinky Hair disease.

Therefore, one of the objectives of this invention disclosure by Kapper is to utilize a copper-assay measurement technique that can accurately measure amounts of copper that are substantially smaller than normal body fluid values.

Another technique available for use as a micromolar copper assay, is the use of X-ray fluorescence spectrometry. Because of the complex theory of this technique, only a brief mention will be made of this technique. However, further reading is suggested and encouraged by searching for these three articles found in the “Silver Platter-Medline Database,” specifically referring to the writings of: A. Wittershagen et al (1997) and B. Aster (1997) contained in the: Spectrochimica Acta Part B Atomic Spectroscopy, and, M. Schmeling (1997) in the: Fresenius' Journal Of Analytical Chemistry.

U.S. Pat. No. 5,973,224 by Fuchs discloses the practice of agricultural foodstuffs enrichment with metallic substances. This invention disclosure by Kapper is clearly an improvement on Fuchs, because it refines, identifies and precisely controls with better accuracy the process and substance distribution in which the plants are to be enriched.

U.S. Pat. No. 6,026,316 by Kucharczyk et al discloses the use of Magnetic-Resonance-Imaging to trace the circulatory and intracranial transport path of drugs through the brain, with an emphasis on the blood brain barrier. This feature of using a radiological technique is particularly relevant to this application disclosure by inventor Kapper, because it supports the feasibility of using nuclear medicine to initialize copper therapy in several invention embodiments. Each embodiment refers to this fundamental invention by Kapper of pharmacologically inducing the dilation of selective arteries or organs, and/or, to precipitate an increased presence of copper serum in specific body areas. These correspond to three separate treatments for several different medical ailments.

The three specific inventions that utilize radiological-vasodilation initialization relevant to this disclosure refer to:

The dilation of the thymus to amplify the immune system, for use as either a vaccination regimen, or, treatment of a current illness, or, for immunoamplification of mammalian embryonic and/or prebirth poultry eggs, similar to a product made by the Embrex corporation.

The dilation of the areas of the brain that are producing excessively high electrical impulses, thereby causing abnormal vasoconstriction to the point of causing seizures.

The radiological technique of confirming the sustained presence of copper in the brain to stop the seizures. The radiological technique used here is the measurement of the general density of copper in the brain. This employs the use of radiology to image what is chemically known as “radioplaques,” for which copper is a member. This means that copper partially blocks the exposure of radioactive X-ray energy to the image recording film by absorbing the radioactive energy. Using this technique, it is possible to measure the amount of copper in the entire brain by the sensitive changes in the density of the X-ray images taken, when compared on a pretreatment and post treatment basis.

Dr. Hirschman of the Adviral Corporation and the American Society of Cytometry has established that the proportion of T-Lymphocyte Helper cells is the biggest factor in HIV/AIDS immunity.

Leavell and Thorup (1976) state that the human body contains between 100-150 milligrams of copper, and that male plasma contains between 89-121 micrograms percent, with female plasma containing between 100-132 micrograms percent.

Jamieson and Zack (1999) discovered that an aged thymus has the ability to rebuild the immune system, and that contrary to popular belief, the thymus does not stop working early in life.

Cappelli-Bigazzi et al (1997) discloses the use of copper-containing blue oxidase of vertebrate plasma to relax rabbit aortic rings.

Stein (1999) discloses the role of copper chloride to improve flow-mediated vasodilation and cholesterol-lowering pharmacological effects.

Medawar (1981) et al discloses the use of retinoids to dilate the thymus for immunotherapy.

Periquet et al (1995) discloses the prevalence of low copper levels in HIV-infected children, and the need for nutritional intervention.

Beach et al (1992) discovered low levels of serum copper in HIV positive patients.

J. E. Sprietma (1997) discloses how copper ions inhibit intracellular HIV replication.

A. Rescigno et al (1997) discloses preparation of reduced tyrosinase from Agaricus bisporus.

The Merck Index of 1983 discloses that tyrosine has the unique ability of passing the blood-brain barrier.

Considine et al (1989) discloses the role of the parts of the brain known as the thalamus and hypothalamus.

Laub and Ruggeri (1988) disclose one of many nuclear magnetic-resonance-imaging techniques that can be used to measure the dilation of arteries in the thalamus and hypothalamus, as manufactured by the Siemens Corporation.

The use of copper-infusion therapy to stimulate the thymus for immunoamplification therapy has been well established. An exhaustive number of documents were found on the “pubmed” website.

Neumann et al (1995) disclose the results of their investigation into a rare plant that has an extraordinarily high concentration of copper. The name of that plant is America Maritima ssp.halleri. This article contains a wealth of information about the concentration of copper in plants. It includes the various techniques needed, along with tips, traps, shortcuts and equipment needed to measure molar copper. Neumann also cites a 1972 article by Reilly regarding the use of amino acids as a copper chelate for copper delivery. This supports the claim by inventor Kapper that his proprietary composition; which consists of coenzyme q10, amino acids, and a vitamin base, artificially sustains the presence of copper in the body, such that the serum-copper level is maintained for an extended time period, longer than the normal metabolism time period. This may also be due to the slowing or suspension of the tyrosine metabolism near the blood-brain barrier, and maintains the presence of copper, because tyrosinase is a copper-containing compound.

In regards to serum copper levels in human blood, as in serum, and not plasma, upon conducting a brief survey of medical textbooks, the inventor has found the following information to be representative, and as from The Textbook Of Medicine, Volume II, by Dr. James B. Wyngaarden and Dr. Lloyd H. Smith, Copyright 1982, W. R. Saunders Publishing, page 2330. Copper serum levels are as follows: Birth to six months; 3.14-10.99 micromoles per liter, or, 20-70 micrograms per dL. A 6 year old child; 14.13-29.83 micromoles per liter, or, 90-190 micrograms per dL. An adult male; 10.99-21.98 micromoles per liter, or, 70-140 micrograms per dL. An adult female; 12.56-24.24 micromoles per liter, or, 80-155 micrograms per dL. A pregnant female at term; 18.53-47.41 micromoles per liter, or, 118-302 micrograms per dL. A pregnant female at term will have between 0.24-0.47 micromoles/d, from a 24 hour urine-sample, and, will have a copper level in their heparin-erythrocytes of between 14.13-23.55 micromoles per liter, or, 90-150 micrograms per dL.

### NOVELTY AND OBJECTIVES

The first and paramount unique feature of this invention is that it uses inorganic chemistry in a field that is completely dominated by organic chemistry, otherwise known as conventional, or, carbon-based chemistry. The active components in this disclosure are inorganic metallic substances. The fields of biochemistry and immunology are completely dominated by organic chemistry.

The second feature and objective of this invention is that it uses a laboratory technique that greatly reduces physician error, increases diagnostic accuracy, and facilitates widespread clinical implementation by utilizing nuclear medicine techniques and radiological equipment. The proper and safe administration of this copper therapy is accomplished by the reading of nuclear X-ray films, as in the visual results of the Magnetic-Resonance-Imaging, or any other nuclear imaging technique, including, but not limited to Positron-Emissive-Tomography, or, related ultrasonic technology. A standardized system of patient care, quality control and liability reduction through the use of visual monitoring by nuclear medicine is much more feasible and desirable, compared to a conventional and non-visual chemical assay test. Specifically, it uses radiology or ultrasonic technology in combination with the experimental substances and compositions by Kapper to:

Confirm that the thymus is enlarged or dilated for immunoamplification.

Confirm the increased density of copper in agricultural plants by use of the proprietary composition and isolated substances contained in this invention disclosure, for the purpose of immunoamplification by agricultural enrichment fertilizer, as suggested by inventor Kapper, and represents an improvement over U.S. Pat. No. 5,973,224 by Fuchs. This particular radiological application uses a relatively low cost and portable X-ray device. These types of devices are implemented here because the high cost of a Magnetic-Resonance-Image system or clinical equipment is not needed to perform this type of copper imaging. Such basic, small, and easily-movable radiological devices are found in dental offices, or, in high-traffic areas, such as crowded emergency rooms or veterinary offices. The radiographic images are taken of the plants before and after treatment of the special metallic-enriched fertilizer, which are added during the initial planting of the seed into the soil. The purpose of using radiology here is to find the best combination and proportion of copper substances and/or proprietary composition(s) to synthesize the maximum copper concentration in the plants. In addition to the use of radiological imaging, a conventional micromolar copper assay is performed on the plants to confirm the greatest levels of copper increase in the plants from the different substance possibilities. The conventional copper assay used here would be similar to U.S. Pat. No. 5,728,539, which was discussed earlier.

Confirm the increase of copper concentration in the brain for treatment of seizures and spasms by taking pretreatment and post treatment nuclear-radiological images to measure the overall density of copper-induced radioplaque in the brain region. Radioplaque refers to a change in the entire contrast of nuclear images of the brain as the concentration of copper increases. The radioplaque images are in addition to the post-radiological maintenance assay for copper in the blood and urine.

The third feature and objective of this invention is that it incorporates a clever and failsafe system to guard against the dangers of copper toxicity. The therapy begins by administering small increases in the dosing of copper substances. At the same time, the proportion of the molar amounts of copper in the blood and urine are documented and tracked. The safe copper dosing level is established as soon as the body areas or arteries have dilated, or, show an increase in “Radioplaque” (copper-imaging) in the brain mapping. Any increase in the copper dosage could then become dangerous. Copper has the potential of being a very toxic substance to the human body. Therefore, to initialize the copper therapy, the patient is administered gradually-increased dosages of copper substances, while, at the same time, the size of the specific areas or arteries in their body are monitored by radiological or ultrasonic imaging. When the areas or arteries have dilated, the clinician measures the amount of copper in moles of the blood and urine by using a copper assay, and establishes the dosage of copper, such that the proportion of serum copper is maintained, as a static proportion in the blood and urine. Note that after the initialization of the therapy to determine the correct proportion of copper in the blood and urine, the nuclear X-ray, and/or, radiological/ultrasonic equipment is no longer used. This represents a significant economic and facilities-utilization benefit, in addition to supporting the mass-marketing of this technique to the general public.

The fourth feature and objective of this invention is that it uses a two-step process to administer therapy. First, the nuclear or ultrasonic imaging is used. Then, therapy is maintained by the use of a copper assay for the blood and urine.

The fifth feature and objective of this invention is that it can be accurately described, classified, and interpreted as nuclear chemical reaction under a wide range of criteria. The therapy utilizes an inorganic metal substance as the active ingredient. It uses the pharmacological benefit of free electrons in the atomic structure of the substances. It uses radiological and nuclear medicine techniques and equipment.

The sixth feature and objective is that the initialization procedure to find the correct and safe maintenance dosage level will automatically adjust and take into account all of the different types of patients. This includes, but is not limited to age, body type, racial or ethnic diversity, and, any existing medical conditions that the patient may have.

The seventh feature and objective of this invention is that it uses compounds, substances, components and elements that are naturally found in the human body. This is very different than the current and general scientific culture, in which the design of hybrid, unnatural or, custom molecules that would never appear in nature are produced by the large drug companies utilizing sophisticated production techniques.

The eighth feature and objective of this invention is that it makes very economic use of nuclear and/or ultrasonic equipment in any setting. In this sense, the invention is easily mass-marketable, is affordable to the patient, and, can be incorporated as a multiple-use, income producing asset to a clinic for long-term financial and research benefit.

The ninth feature and objective of this invention is that it forms a scientifically acceptable and investigational routine to develop and quickly refine the clinical and laboratory technique to utilize the beneficial and pharmacological dosing of copper, and the related phenomena of:

Using the proprietary substances and compositions by inventor Kapper to sustain the presence of copper in the brain and selected areas of the body by taking advantage of altering the tyrosinase-tyrosine metabolism, and, at the same time utilizing the property of tyrosine to cross the blood-brain barrier (Merck Index 1983).

As an improvement over previously discussed U.S. Pat. No. 5,973,224 by Fuchs, as in immunoamplification by metallic-fertilizer enrichment of agricultural foodstuffs.

Using the proprietary substances and compositions by inventor Kapper to increase the serum copper level in the brain as a therapy regimen for seizures, spasms, and any medical disorder that is induced by excessive electrical activity in the brain, such that the abnormally high electrical gradients are reduced. Using the proprietary substances and compositions by inventor Kapper to dilate specific arteries and areas in the body, such as in the thymus.

Using the proprietary substances and compositions by inventor Kapper as a vaccination regimen, and/or, a treatment for an illness that has already appeared, and/or, as an embryonic-prebirth treatment for a mammal, and/or, a poultry/eggshell administered therapy.

The tenth feature and objective of this invention is that it has none of the terrible disadvantages of organic chemistry, otherwise known as conventional, or, carbon-based chemistry. This invention utilizes nuclear chemistry in a clinical radiology setting. The radiological identification of the transport system related to the inorganic metallic substances in both human body fluid and in the solid body metabolism can be easily identified by utilizing radiological equipment. On the other hand, when an organic molecule is used as a pharmacological therapeutic, there is a much higher probability that the organic molecule will interfere with normal body processes, and will precipitate an abnormal, harmful, or life-threatening reaction. This is because organic molecules typically have many reactive or receptive sites in and around the molecule structure that can collide or combine with organic molecules in the body, such that they could alter organic processes in the body. These harmful effects are not easily predictable, are not well-studied, and are not easily identifiable in the body. The terrible problem is that the practitioner will not be aware that there is a problem until the outward or visible symptoms begin to manifest. This kind of delay represents huge liabilities to the practitioner and patient. Furthermore, and, unlike inorganic nuclear chemistry, there would be a larger amount of time needed to identify the problems with an organic pharmacologic. This is because it is more difficult to find the source of the conflict. In other words, in evaluating the issue of complications resulting from the use of therapy derived from an inorganic-nuclear molecule compared to an organic molecule, there are many more variables and a much greater possibility of conflicts presented by using a conventional organic pharmacologic in the body. This is because unlike nuclear chemistry, the practitioner does not have benefit of using nuclear radiology to quickly identify the transport mechanism of the nuclear therapeutic particles, or, pharmaceutically-induced vasodilation, and related increased presence of copper.

The eleventh feature and objective of this invention is to document the benefit of continuing studies that would reinforce and perpetuate the principles outlined in this invention disclosure. It would certainly be of great benefit to medical science if the most common agricultural-edible plants found on the planet were to be reclassified on the basis of how the various plant metals are radiologically-imaged within the particular plant structures and leafs. Another purpose of this would be to identify the particular metabolism-mechanism(s) within the plants. The purpose and objective of such a huge undertaking is that it would greatly expedite and unify the large amount of scattered research currently being performed by private companies attempting to harness the benefit of metallized-substance therapies derived from plants. Furthermore, such a classification system for plants would create a “common-platform” for researchers from the many different areas within natural science and molecular biology. The basic information that would be obtained to classify a plant would be:

A nuclear image that would display the location and density of metal atoms within the entire plant, including the body and individual exposures of single leafs.

A copper assay to indicate the overall concentration of copper within a specific plant in comparison to other plants.

Identify what particular parts of different plants or leafs have the greatest concentration of copper, as in different species and the individual plant names. Conduct tests on all plants to see which particular plant varieties are the easiest to enrich by using the techniques suggested in this invention disclosure. The purpose and objective is to find out what plants respond the most to the concept of amplifying the immune system by fertilizer-enrichment. It may be necessary to genetically alter a plant to increase the copper-carrying potential of the plant. Such a plant that could be considered for genetic cross-breeding to other plants is the plant known as Armeria Maritima ssp.Halleri. A description of this plant by Neumann and Nieden is contained in the prior art section of this application. Basically, this plant has an extraordinary high concentration of copper. It grows near a volcano. The article attempts to explain how it is possible that this species of plant could survive and reproduce in a geographic area that has such a high concentration of copper.

### DESCRIPTION OF INVENTIONS

Invention I amplifies the immune system by dilating the thymus, thereby causing an increase in the T-lymphocyte helper cells, and is an improvement over U.S. Pat. Nos. 5,234,683, 5,114,708, and 4,215,137.

Invention II amplifies the immune system by enriching agricultural foodstuffs, and is an improvement over U.S. Pat. No. 5,973,224.

Invention III is based on injecting the experimental substances into poultry eggs.

Invention IV is a treatment for seizures, and can be used in cases where the seizures are caused by: (1) a copper deficiency in the brain, as evidenced by low copper levels found in the brain of autopsied epileptics, or, (2) excessive electrical impulses in the brain, or, (3) excessive vasoconstriction in the brain. (4) Other conditions, such as tourettes syndrome.

Invention V determines the propensity of immunoamplification for various agricultural plants.

### PROPRIETARY COMPOSITION COMPONENTS

The following formula sheet for the oral proprietary composition is strictly a general guideline for individual components. It shall be clearly understood that the volume of individual components may vary greatly, and that these variations shall clearly fall into the scope and intent of the invention for both oral and injectable administration. The oral formulation sheet should serve as a guide if an injectable embodiment is desired. The molar amounts given by injection should roughly correspond to the proportional amounts of the individual components from the oral compound.

This composition consists of: A copper-containing or metallic substance, a multivitamin, CoEnzyme Q1 to Q10 and Amino acids.

Copper or metallic substances can be very toxic. Therefore, one should observe the toxic limits known in literature, and should use the smallest amount possible to achieve the desired invention embodiment.

Multivitamins are a low toxic threat. The toxic levels of vitamins have been well established. A relatively large amount of multivitamins can be ingested.

CoEnzyme(s) Q1 to Q10 are a very low toxic threat. A relatively large amount of CoEnzyme(s) Q can be ingested, including much more than what is specified on the formula sheet.

Amino acids can be harmful and toxic.

### DOSING OF THE PROPRIETARY COMPOSITION COMPONENTS

15 grams of powdered “All-In-One” nutrient powder (non-yeast).

3 grams of powdered Tryptophane.

2 grams each of powdered amino acids Tyrosine, Taurine, Lysine, L-Phenylalanine, and D-Phenylalanine.

2 grams of powdered Niacinamide (Vitamin).

1.5 grams of powdered Niacin (Vitamin).

1 gram each of powdered amino acids L-Methionine and Histidine.

500 milligrams of powdered Arginine.

250 milligrams of powdered GABA.

180 milligrams of powdered CoEnzyme Q10.

13-17 ounces of non-artificial orange juice.

2 teaspoons of liquid protein, such as the tradename, “Cher-Amino.”

What is claimed is:

1. A method for amplifying the effect of the autoimmune system or increasing the T-lymphocyte helper cells for achieving vaccination or as a treatment for an existing and active disease in animals, including humans, comprising:
enlarging the thymus gland by administering a series of gradually increasing dosages of a copper containing or any metallic substance;
monitoring the size of the thymus to detect the maximum dilation;
measuring the micromolar amount of copper or metallic substance in the blood and urine by utilizing a micromolar assay for the copper or metallic substance;
and controlling the amount of copper containing or metallic substance administered such that the proportional quantity of micromolar copper or metallic substance in the blood and urine is maintained.

2. The method of claim 1, wherein the monitoring step is done following each increase in copper or metallic substance dosage.

3. The method of claim 2, wherein the monitoring is determined by using at least one of nuclear X-ray, radiological, MRI, PET or ultrasonic imaging.

4. The method of claim 3, wherein the use of nuclear X-ray, radiological, MRI, PET or ultrasonic imaging is utilized only for the initial administration of the copper containing or metallic substance, and after maximum thymus dilation is observed, future administration of the copper containing or metallic substance dosage is achieved solely by use of a micromolar copper or metallic substance assay for the blood and urine.

5. The method of claim 1, wherein the enlarging the thymus gland is achieved by injecting the copper containing or metallic substance directly into the thymus gland.

6. The method of claim 1, wherein the enlarging the thymus gland is achieved by administering the copper containing or metallic substance through an artery or vein.

7. The method of claim 1, wherein the administration of the copper containing or metallic substance is achieved by ingesting plants having an elevated concentration of a copper containing or metallic substance.

8. The method of claim 7, wherein the plants contain an elevated concentration of a copper containing or metallic substance by enriching the roots and soil surrounding said roots during seeding and/or feeding of said plants with a copper containing or metallic substance.

9. A method of treating a patient suffering from Tourette's syndrome, seizures, spasms, excessive EEG pulses, vascular-vasoconstrictive headaches, epilepsy or any copper or metallic deficiencies in the brain by administering a copper containing or metallic substance to the patient in a series of gradually increasing dosages until the X-Ray film or any radiological device becomes capable of measuring the contrast from radioplaque-copper;
measuring the micromolar amount of copper or metallic substance in the blood and urine;
controlling the amount of the copper containing or metallic substance administered such that the proportional quantity of micromolar copper or metallic substance in the blood and urine is maintained.

10. The method of claim 9, wherein the detection is by symptom relief.

11. A method of immunoamplification in a prebirth state, comprising:
injecting a copper containing or metallic substance into a poultry egg;
increasing the dosage until immunoamplification is accomplished.

12. A process of determining the propensity and suitability of agricultural plants to harness the immunologic benefits of being enriched by adding metallic substances comprising:
taking a nuclear image that displays the location and density of metal atoms within an entire plant, including the body fiber, roots and leaf;
conducting a micromolar copper or metallic assay to indicate the overall concentration of copper or metallic substance within a specific plant in comparison to other plants;
enriching the copper or metallic content of the plant by administering a copper containing or metallic substance to the plant root or soil surrounding said roots during seeding and/or feeding of said plants.

13. The process according to any one of claims 1, 7, 9, 11, and 12, wherein the copper containing or metallic substance is selected from the group consisting of:
(a) tyrosinase
(b) methyl blue
(c) a mixture of tyrosine, CoEnzyme Q10 or CoEnzyme Q1 to Q9, tryptophane, aromatic amino acids, phenylalanine, other amino acids and a vitamin base, any copper containing substance; and mixtures thereof;
(d) any metallic atom

14. A composition comprising a mixture of at least one amino acid, CoEnzyme Q10 or CoEnzyme Q1 to Q9, a multivitamin composition and a copper containing substance.

15. The composition of claim 14 where the copper containing or metallic substance is selected from the group consisting of tyrosinase, methyl blue, or any metallic substance.

16. The composition of claim 14 further comprising a liquid carrier selected from the group consisting of liquid amino acids, liquid vitamins and mixtures thereof.

17. The composition of claim 16 further comprising phenylalanine, tyrosine and tryptophane.

18. A process for artificially sustaining the level of copper or metallic substance beyond the normal metabolism time in agricultural plant leafs or fibers, the human or animal body parts of the brain, brain fluids, arteries, veins or thymus comprising: administering a copper containing or metallic substance to the plant or patient in the presence or in combination with the composition in claim 14.
